(19) United States
(12) Patent Application Publication
(10) Pub. No.: US 2007/0253987 A1
(43) Pub. Date: Nov. 1, 2007

(54) Novel pigments for wrinkle-hiding cosmetic applications

(76) Inventors: Mark E. Wozniak, Bel Air, MD (US); Duen-Wu Hua, Edgewood, MD (US); Karl W. Gallis, Perryville, MD (US)

Correspondence Address: J M HUBER CORPORATION, 333 THORNALL STREET, PATENT DEPARTMENT, EDISON, NJ, 08837-2220, US

(21) Appl. No.: 10/978,612

(22) Filed: Nov. 1, 2004

(51) Int. Cl. A61K 8/02; A61Q 19/00

(52) U.S. Cl. 424/401

(57) ABSTRACT

Unique pigments that exhibit excellent light diffusion when applied to a person's skin within an appropriate medium as well as acceptable feel during such application are provided. To be most efficacious for this application, such pigments should meet specific criteria being a proper particle size, having irregular structure (i.e. not smooth), and a non-spherical shape. In such a manner, the subject pigments effectuate the desired ability to diffuse light when applied to skin (in a film of acceptable cosmetic composition) such that optical blurring takes place thereby preventing the appearance of wrinkles. The shape, particle size and irregularity of the pigments are necessary to permit maximum light diffusion effects. The particle size and particle size range also necessarily provides a feel that is sufficiently soft for skin applications. Specific types of pigments, as well as cosmetic formulations including such novel materials are also encompassed within this invention.

### FIELD OF THE INVENTION

This invention relates to unique pigments that exhibit excellent light diffusion when applied to a person's skin within an appropriate medium as well as acceptable feel during such application. To be most efficacious for this application, such pigments should meet specific criteria being a proper particle size, having irregular morphology (i.e. not smooth, and non-spherical shape). In such a manner, the subject pigments effectuate the desired ability to diffuse light when applied to skin (in a film of acceptable cosmetic composition) such that optical blurring takes place thereby preventing the appearance of wrinkles. The particle size and irregularity of the surface and the shape of the pigments are necessary to permit maximum light diffusion effects. The particle size and particle size range also necessarily provides a feel that is sufficiently soft for skin applications. Specific types of pigments, as well as cosmetic formulations including such novel materials are also encompassed within this invention.

### BACKGROUND OF THE INVENTION

There is an understood if not perceived need for skin surface modifying compounds and compositions to provide aesthetic qualities to a person's appearance. As a person increases in age, his or her skin will become altered through stretching and relaxing, and exposure to environmental conditions thereby creating crevices therein typically referred to as wrinkles. As the existence of wrinkles is a fair indicator of a person's age and/or skin condition, particularly when such wrinkles are present within the person's facial region, should the subject person wish to prevent the outward appearance of such age-signifying wrinkles, then blurring, covering up, and/or preventing such outward wrinkle appearances is highly desirable. For many years, makeup, foundation, and like cosmetic formulations, have been utilized to improve a person's appearance on a temporary basis. However, many users complain that these cosmetic products do not sufficiently reduce the appearance of their wrinkles and fine lines. There is thus a definite want and/or need within the cosmetic industry to provide formulations that, upon contact and application to skin provide a smooth and even-looking skin tone to the user. Some examples of prior attempts at wrinkle hiding cosmetic formulations include U.S. Pat. No. 5,082,660 to Ounanian et al., U.S. Pat. No. 4,255,416 to Gillespie, U.S. Pat. No. 4,965,071 to Kawan, U.S. Pat. No. 4,362,715 to Strianse et al., U.S. Pat. No. 4,659,562 to Arraudeau et al., 6,630,131 to Chevalier et al., and U.S. Pat. No. 5,587,170 to Caisey et al., as well as U.S. Pat. Appl. Publication US2003/0007985 to Chevalier et al.

The prominent appearance of lines and wrinkles on the skin is due to optical geometry. In terms of a person's skin, diffuse reflectance occurs readily, but to differing degrees. When the surface of the skin is smooth, light is absorbed, reflected and scattered off the skin and is observed as a color according to how much light is absorbed and scattered. To the contrary, however, the intensity of the light reflected back to the eye from wrinkles on the skin surface is less than that from normal skin and, as a result, the eye will perceive the wrinkled skin regions as darker and thus more noticeable. An increase in the degree of diffuse reflectance would help to modify the perceived appearance of wrinkled skin since the ability to scatter light in greater directions would prevent the eye from clearly viewing the skin surface as it actually exists.

It is generally agreed upon that the following characteristics are necessary for a cosmetics formulation to exhibit efficient wrinkle hiding. The total transmittance of the cosmetics film needs to be high, so that it is not visible when applied to the skin. The diffuse transmittance component of the total transmittance should be as high as possible, so that incoming light is distributed evenly across the skin. Light absorbed by the film and also light reflected by the film should be minimized. If possible, any light that is reflected by the film should be a diffuse and not a specular reflection. A UV-Visible spectrophotometer equipped with an integrating sphere detector can be used to measure these optical properties, as first described by Nakamura, et. al, (“Blurring of Wrinkles Through Control of Optical Properites” Nakamura, Naoki; Takasuka, Yutaka; and Takatsuka, Isamu. Preprints of the XIVth I.F.S.C.C. Congress, Barcelona 1986, Vol 1, 51.) Nakamura found that thin films comprised of a pigment and an oil that exhibited the highest diffuse transmittance values were most effective at blurring wrinkles when applied to the skin.

To prevent the appearance of lines and wrinkles, makeup or foundation formulations have been modified to incorporate pigments which manipulate the light, and modify the appearance of the surface to which it is applied. Light manipulation, therefore, has become an important development in makeup products. To mask the appearance of wrinkles on the skin, the main goal of optically diffusing pigments is to scatter the incident light more evenly across the surface of the skin in order to minimize the quantity of light lost in a wrinkle, thus making wrinkles appear less visible. An optical light diffusing pigment can be, for example, titanium dioxide, which reflects light. As a result the line is masked and not highlighted because of the reflective nature of the titanium dioxide. However, this method has been found to be undesirable because titanium dioxide particles generally exhibit indices of refraction vastly different from those of common cosmetic formulations, thereby making the target cosmetic formulation too opaque for sufficient transmittance of light to occur. As a result, the formulation would invariably appear white when applied to a user's skin, rather than permitting optical blurring with a skin-tone coloration. Avoidance of such a white coloration is therefore required for proper cosmetic benefits to be provided.

Thus, other pigments, particularly those of the in the R.I. range of 1.4 to 1.6, to provide non-whitening effects, have been utilized to a great extent for wrinkle hiding with a modicum of success. Such refractive index properties can be modified as well through doping base materials, such as silica doped with iron oxide or aluminum oxide, as an example, or different metal oxides may be mixed together to impart desired coloring and/or toning effects on the skin. Generally, as well, such pigments have also been modified morphology-wise to be necessarily spherical in shape in order to provide what was previously thought of as required soft and supple feel on the target skin surface. The spherical shape has been considered imperative because, as was believed in the past, a rough-edged material would exhibit a scratchy or at least an uneven skin surface feel when and after being applied. Thus, in order to provide sensory benefits, utilization of smooth, spherically shaped pigments has been followed. Problems have been realized with such materials, however, because of inadequacies in formulating proper cosmetic compositions for wrinkle hiding effects. Proper levels of oil components in proportion to these particulate spherical pigments have been difficult to determine without losing wrinkle hiding capabilities, particularly considering the costs of including such properly produced spherical pigments. Although higher loadings of such pigments may provide beneficial wrinkle-hiding effects overall, the costs to provide such beneficial properties and formulations are sufficiently high to prevent such a result.

Such spherically shaped pigments have suffered other noticeable drawbacks as well. Most importantly, the level of optical blurring provided by such spherical materials at practiced loading levels is not as high as necessary for long-term, effective wrinkle-hiding results. The degree of cover-up desired for such cosmetic formulations is quite high and long-lasting; to date, even the most popular spherical pigments provided within the industry are limited in terms of sufficiently and consistently hiding wrinkles and/or lines. The costs associated as well with producing such spherical materials create costs that are ultimately passed on to the consumer. Furthermore, other types of pigments, such as interference pigments (such as, pearlescent pigments), crystalline types of colored pigments, nano-particulates on platy substrates, and the like, have claimed effectiveness at hiding skin surface wrinkles and other blemishes; however, such materials have proven, as with the spherical types commonly used and noted above, extremely expensive to manufacture and incorporate at effective levels within cosmetic. Thus, there is a need to provide pigments and cosmetic formulations that permit excellent wrinkle-hiding without sacrificing skin feel and without increasing manufacturing and/or formulating costs and/or complexities. Again, to date, such improved materials have not been provided within the cosmetic industry.

### OBJECTS AND SUMMARY OF THE INVENTION

It is thus an object of the invention to provide a predominately non-spherical pigment that provides effective light diffusion and optical blurring when applied to a user's skin while also providing a skin tone appearance and exhibiting a sufficiently soft feel. Another object of the invention is to provide a cosmetic formulation that includes such a predominately non-spherical pigment material. Yet another object of the invention is to provide a silica-based pigment and cosmetic formulation thereof that exhibits the same characteristics.

Accordingly, this invention encompasses a method of providing optical blurring of a skin surface comprising the steps of a) providing a cosmetic formulation for application to skin comprising an appropriate cosmetic vehicle and at least one type of pigment, wherein said pigment is a material that is comprised of a plurality of particles that are predominately non-spherical and three-dimensional in nature, wherein the median particle size of such plurality of particles is from 1 to 20 μm, preferably from about 1 to 10 μm, more preferably from about 1 to 5 μm, and most preferably from about 1 to 3 μm, and wherein said plurality of particles exhibits an average oil absorption capacity of from about 10 to 300 ml/100 g, preferably from about 100 to 240 ml/100 g, and b) applying such a cosmetic formulation to a selected area of skin. Also encompassed within this invention is the cosmetic formulation defined above in solid, gel, paste, lotion, cream, spray, loose powder, pressed powder, or liquid form. Furthermore, this invention encompasses a method as defined above wherein the cosmetic formulation comprises the necessary vehicle as noted above and wherein the plurality of pigment particles present therein are silica and/or silicate-based.

For purposes of this invention, the term “non-spherical” is intended to indicate that the particles exhibit three-dimensional structures and they cannot be defined by a radius of fixed length.

### DETAILED DESCRIPTION OF THE INVENTION

In terms of the pigment material present within the invention, any particulate material that meets the limitations noted above can be used. Thus, as long as the particulate material is irregular in shape and rough on its surface and is three-dimensional (and thus when viewed under a microscope at proper levels, the appearance of the material is not a shape that would result in specular reflection, and thus appears uneven in at least three planes of geometry), exhibits the proper median particle size (from about 1 to 20 μm; more preferably from about 1 to 10; more preferably from about 1 to 5; and most preferably from about 1 to 3), and exhibits the proper oil absorption (from about 50 to 300 cc/100 g, more preferably from about 100 to 240), then the material meets the defined invention. The presence of predominately non-spherically shaped pigment particles has been found to maximize the required light diffusion and optical blurring for the inventive wrinkle-hiding compositions. A non-spherical pigment with an irregular surface should scatter light more efficiently than a spherical particle with a smooth surface, although spherical materials do provide a certain degree of beneficial light-scattering. In such a manner, the skin wrinkles to which such a pigment is applied will not be easily viewed, but will be blurred.

The proper pigment loading which can be determined from the oil absorption of the pigment and knowledge of the cosmetic formulation and the refractive index of both is necessary to impart a proper skin tone result. If the loading level is not properly matched by using knowledge of the pigments oil absorption and cosmetic formulation, then the aforementioned deleterious whitening effect will occur or the skin will appear shiny, thereby defeating the purpose for which the pigment is needed. Furthermore, the importance of providing a very narrow range of particle sizes is very important to provide an evenness to the blurring results, as well as permit even application, and smooth and soft feel to the skin during application. Surprisingly, it has now been found that not only can non-spherical pigments provide the necessary light diffusion and reflectance needed for improved wrinkle-hiding effects, but the inclusion of sufficiently small, yet highly effective optically blurring materials, within a narrow particle size range provides excellent feel characteristics. The feel results are sufficiently acceptable for skin contact applications.

As such, any pigment material that is modified to meet these requirements is considered encompassed within this invention, particularly those that are amorphous in nature. Thus, although one potentially preferred embodiment is precipitated silica or silica gel pigments, other materials, such as metal oxides, including, without limitation, titanium dioxide, zinc oxide, iron oxide, aluminum oxide, and any common variations on such oxide compounds, metal silicates, including, again without limitation, aluminosilicate, magnesium aluminosilicate, calcium silicate, particularly types that are coated in order to prevent pH problems when included within cosmetic formulations, clays, such as, without limitation, kaolin, montmorillonite, and the like, metal carbonates, including, without limitation, calcium carbonate (ground, or precipitated), metal hydroxides, including, without limitation, aluminum trihydrate, boehmite, and the like, hydrotalcites, metal phosphates, and synthetic pigments like those made from polyacrylates or polycarbonates.

Although the preferred pigment materials are non-spherical in structure, within the cosmetic formulations possibly including such inventive pigments, any type of pigment, spherical, flat, or otherwise, including other non-spherical types, may be added for beneficial coloring, shading, etc., if desired.

The present invention also includes methods of reducing the appearance of fine lines and wrinkles on the skin by blurring their ability to be observed. The pigment materials (such as a potentially preferred, non-limiting, precipitated silica/silicate pigment, as presented within the preferred embodiments below) are blended together (either alone or with other pigments, as noted above) based on their ability to manipulate light and provide a natural skin tone color. This combination of pigments when applied to the skin surprisingly produces a diffused reflection of light such that the observer views a smooth and flawless skin surface. The appearance of lines, wrinkles, minor deformations and minor discolorations on the skin are less visible. The compositions of the present invention optimize the optical diffusion of light and cause the appearance of lines, wrinkles, deformations and other discolorations to substantially vanish. As a result, the natural skin color is seen as smooth and flawless, and the coverage is sufficient to reduce the appearance of redness and other skin discolorations.

Within the cosmetic formulation, the inventive pigment materials are present in an amount of about 0.01 to about 20 percent by weight of the composition; preferably from about 0.1 to about 15 percent, more preferably from about 0.5 to about 10, and most preferably from about 2 to about 7. The other possible pigment materials may be added in like amounts if desired. Furthermore, if other pigments are added to the cosmetic formulation for aesthetic or other purposes and such materials absorb oil, the quantity (loading level) of wrinkle hiding pigment will most likely require adjustment downward to achieve maximum wrinkle blurring effects.

Other coloring or shading components may be added as well within the cosmetic formulations, such as mica, bismuth oxychloride, sericite, alumina, aluminum, copper, bronze, iron oxides, ultramarine violet, ultramarine pink, manganese violet, carmine, organic dye lakes and salts, ferric ferrocyanide, ferric ammonium ferrocyanide, chromium oxide, chromium hydroxide, silver or silica (for shimmering effects, for instance), and other like materials. Certain spherical powders that achieve an optical blurring effect can be added as well, including calcium aluminum borosilicate, PMMA, polyethylene, polystyrene, methyl methacrylate crosspolymer, nylon-12, ethylene/acrylic acid copolymer, boron nitride, Teflon, or silica Other examples of possible additives include soft focus materials incorporated include products available from Ikeda (such as VELVETVEIL®) a mica coated with spherical silica beads (such as SOFT VISION®, from Sunjin Chemical of Fort Lee, N.J.), a mica coated with silica beads and further coated with TiO₂ (such as GANZPEARLS® GSC-30SR and GSC-30MC, from Preserve, Inc. of Piscataway, N.J.), a sericite and crosslinked polystyrene, and a mica and crosslinked polystyrene, respectively.

Further cosmetic composition additives include standard interference pigments. Interference pigments are defined as thin platelike layered particles having a high variation of refractive index, which, at a certain thickness, produce interference colors, resulting from the interference of typically two, but occasionally more, light reflections, from different layers of the plate. The most common examples of interference pigments are micas layered with about 50 to 300 nm films of TiO₂, Fe₂O₃, or Cr₂O₃. Such pigments are often pearlescent. Pearl pigments reflect, refract and transmit light because of the transparency of pigment particles and the large difference in the refractive index of mica platelets and, for example, the titanium dioxide coating. The reflected light appears as a luster, because light is split by pigment particles at different depths to create a multidimensional shimmer, commonly referred to as pearlescent. The pigments are very reflective, and as mentioned above, are not suitable alone in a makeup product designed to resemble the natural skin tone.

Useful interference pigments are available commercially from a wide variety of suppliers, for example, Rona/E.M. Industries (TIMIRON™ and DICHRONA™), Presperse (FLONAC™), Englehard (DUOCHROME™), and Kobo (SK-45-R and SK-45-G). Further examples of interference pigments are Flonac MS-30C, mica treated with TiO₂ and iron oxide (yellow), and MU-10C, mica treated with TiO₂ (white). Preferably, interference pigments of different colors or types are combined in the present invention to blend an appropriate shade or intensity of color to match the natural skin tone. The size of the interference pigment can be varied, depending upon the effect desired. Generally, a smaller pigment is less pearly, and therefore preferred, as the larger pigments will confer a substantial amount of sparkle. A useful size range of the interference particles is from about 1 to about 200 μm and preferably is about 3 to about 100 Sun. The interference pigment is used in an amount of from about 0.05 percent to about 50 percent by weight. However, in most types of products, the amounts of interference pigment will range from about 0.5 percent to about 20 percent, the lower end of the range being used in products using no non-interference pigments or lightly pigmented products, and the higher end of this range being used in more heavily pigmented products. Also, when the vehicle is a water-in-oil or water-in-silicone emulsion, it may be desirable to coat the interference pigments with a hydrophobic coating, or other suitable coating to facilitate wetting out. Thus, pearl pigments may be uncoated or coated.

Inorganic pigments, organic pigments, or a combination thereof can be used to impart color and tone to a user's skin. Examples of useful inorganic pigments include, without limitation, talc, lecithin modified talc, zeolite, kaolin, lecithin modified kaolin, titanium dioxide, zinc oxide, and mixtures thereof. Metal oxides, particularly iron and titanium oxides, and kaolin are preferred non-interference pigments in the composition of the invention. In addition to providing color to match the color of the skin, titanium dioxide, zinc oxide, and iron oxide function as particulate inorganic sunscreens.

Organic pigments can include natural colorants and synthetic monomeric and polymeric colorants. Exemplary are phthalocyanine blue and green pigment, diarylide yellow and orange pigments, and azo-type red and yellow pigments such as toluidine red, litho red, naphthol red and brown pigments. Also useful are lakes, which are pigments formed by the precipitation and absorption of organic dyes on an insoluble base, such as alumina, barium, or calcium hydrates, and toners, such as salts of organic dyes. Particularly preferred lakes are primary FD&C or D&C Lakes and blends thereof. Water soluble colorants (such as FD&C Blue #1), oil soluble colorants (such as D&C Green #6), and stains, such as bromo dyes and fluorescein dyes can also be employed. One particularly prevalent toner is D&C Red #7, calcium salt. The amount and type of the non-diffusing pigment used will vary depending upon the nature of the final product and the desired intensity of color; generally, however, the amount of non-diffusing pigment will be about 1 to about 30 percent, and preferably about 1 to about 5 percent, by weight of the total composition. In addition, microfine particulate pigments can be used at somewhat higher levels than those of normal particle size without significantly increasing the level of opacity of the composition on the skin can.

The inventive pigment materials can be used in any type of skin treatment or makeup product. Skin treatment products include lip products, acne treatments, moisturizers, anti-aging products, lifting treatments, cellulite treatments and eye treatments. The makeup products of the invention include, but are not limited to, foundations, blushes, pressed or loose powders, concealers, bronzers, eyeshadows, eyeliners, lipsticks, and lipglosses. The products of the invention can take any form which is typical of cosmetic products, for example, hot pour formulations, water-in-oil emulsions, oil-in-water emulsions, gels, sticks, sprays, anhydrous formulations, and silicone-based liquid formulations. There is no limitation on the type of vehicle that can be employed. In particular, the preferred identity of the vehicle will be largely controlled by the type of product into which the components are to be incorporated. For a liquid foundation, for example, a silicone or oil-in-water emulsion is preferred for aesthetic reasons, and although the oil portion of the vehicle can be any which is typically used for this purpose, it is preferred that the oil component comprise a silicone oil, either volatile or non-volatile. The non-aqueous phase of make-up emulsions can contain silicones as noted, however they may also contain any or all of the following: synthetic mono, di and tri esters, triglycerides, perfluoroethers, squalane, mineral oil, synthetic polymers, bentonite clay, modified starches, fatty alcohols, fatty acids, alkyl polyethoxylated esters and ethers, castor natural oils among others. Preferably, the present invention is used in an emulsion or liquid form.

The formulation also can comprise other components that may be chosen depending on the carrier and/or the intended use of the formulation. Additional components include, but are not limited to, water soluble sunscreens (such as Eusolex 232); sunscreens (such as octyl methoxycinnamate, camphor derivatives, cinnamates, salicylates, benzophenones, triazines, PABA derivatives, diphenylacrylate derivatives, and dibenzoylmethane derivatives.); antioxidants (such as BHT); chelating agents (such as disodium EDTA); emulsion stabilizers (such as carbomer); preservatives (such as methyl paraben); fragrances (such as pinene); humectants (such as sorbitol, glycerine, and the like); waterproofing agents (such as PVP/Eicosene copolymer); water soluble film-formers (such as hydroxypropyl methylcellulose); oil-soluble film formers (such as hydrogenated C-9 Resin); moisturizing agents, such as cholesterol; cationic polymers; anionic polymers; pigment wetting agents; vitamins (such as tocopherol); and the like.

The compositions can also encompass one or more active components, and as such can be either cosmetic or pharmaceutical compositions. Examples of useful actives include, but are not limited to, those that improve or eradicate age spots, keratoses and wrinkles, analgesics, anti-acne agents, antibacterials, antiyeast agents, antifungal agents, antidandruff agents, antidermatitis agents, antipruritic agents, antiemetics, antihyperkeratotic agents, anti-dry skin agents, antiperspirants, antipsoriatic agents, antiseborrheic agents, antiaging agents, antiwrinkle agents, sunscreen agents, skin lightening agents, depigmenting agents, vitamins, tanning agents, or sunscreens. More specific examples of useful active agents include retinoids such as retinol, and esters, acids, and aldehydes thereof; ascorbic acid, and esters and metal salts thereof, tocopherol and esters and amide derivatives thereof; shark cartilage; milk proteins; alpha- or beta-hydroxy acids; DHEA and derivatives thereof; and benzoyl peroxide, and like materials.

### Preferred Embodiments of the Invention

The invention is further illustrated by the following non-limiting examples:

### EXAMPLES

The inventive wrinkle hiding pigments evaluated in the following examples were prepared as described below.

Pigment A

322 L of an aqueous solution of sodium sulfate (11.4%) was added to a 400-gallon reactor and was heated to 60° C. with stirring at 75 RPM. Once 60° C. was reached, sodium silicate (20.0%, 2.65 MR) was added for 4 minutes at 12.24 L/min. Separately, a solution of aluminum sulfate (alum) was prepared by reacting 1343 g kaolin with 1463 g of 95% sulfuric acid in 3150 ml of water under agitation in an autoclave at 186° C. for 10 hr. The reaction mass was discharged, cooled and diluted with water to a concentration of 0.3 g alum/ml. After 4 minutes had passed, sodium silicate (20.0%, 2.65 MR) and an aqueous alum solution (0.3 g/ml alum) were simultaneously added at rates of 12.24 L/min and 5.74 L/min, respectively, for 35 minutes. After a simultaneous addition time of 35 minutes, the flow of silicate was stopped and the pH was adjusted to 5.0 with continued addition of the alum solution at 5.74 L/min. Once pH 5.0 was reached, the batch was digested for 15 minutes at 70° C. It was then filtered, washed and was spray dried.

Pigment B

Pigment B was prepared by hammer milling Pigment A to a median particle size of approximately 8 μm.

Pigment C

Pigment C was prepared by air milling Pigment A to a median particle size of approximately 2 μm.

Pigment D

420 L of an aqueous sodium sulfate solution (11.4%) was added to a 400-gallon reactor and was heated to 70° C. with stirring at 75 RPM. Once 75° C. was reached, 4 L of magnesium hydroxide slurry (50% solids) was added. An aqueous solution of alum (48.0%) was then added at 3.22 L/min for 3.5 minutes. After 3.5 minutes, the flow of alum was stopped and the batch was allowed to digest for 2 minutes. After the 2 minute digest time, sodium silicate (31.0%, 2.5 MR) and alum (48%) were added simultaneously at rates of 7.42 L/min and 3.22 L/min, respectively, for 35 minutes. After the 35 minute simultaneous addition time, the flow of silicate was stopped and the pH was adjusted to 6.5 with continued addition of alum at 3.22 L/min. Once pH 6.5 was reached, the flow of alum was stopped and sodium silicate (31.0%, 2.5 MR) was added for 4 minutes at 7.42 L/min and was then stopped. The batch was digested for 15 minutes at 75° C. After the digestion time, the batch was filtered and washed. Prior to spray drying, the pH of the batch was re-adjusted to 7.0 with the addition of a small amount of alum. After spray drying, the product was then air milled to a median particle size of approximately 1 μm.

Pigment E

278 L of sodium silicate (13.3 %, 2.65 MR) was added to a 400-gallon reactor and was heated to 65° C. Once 65° C. was reached, sodium silicate (13.3%, 2.65 MR) and a sulfuric acid/alum solution (prepared by mixing 250 L 11.4% sulfuric acid with 202 L 15.4% alum) were added simultaneously at the rates of 9.7 L/min and 6.3 L/min, respectively, for 47 minutes. After 47 minutes had passed, the flow of silicate was stopped and the pH was reduced to 5.0 with continued addition of the alum/acid solution at 6.3 L/min. Once pH 5.0 was reached, the solution was digested for 10 minutes at 65° C. It was then filtered, washed to a conductivity of 1500 μS and spray dried. The dry product was air milled to a median particle size of approximately 1 μm.

Pigment F

553 L of sodium silicate (13.3%, 2.65 MR) was added to a 400-gallon reactor and was heated to 85° C. Once 85° C. was reached, sulfuric acid (11.4%) was added at a rate of 5.8 L/min for 42 minutes. After 42 minutes had passed, the flow of sulfuric acid was stopped and sodium silicate (13.3%, 2.65 MR) was added at a rate of 11.1 L/min for 10 minutes. After 10 minutes had passed, the flow of silicate was stopped and the pH was adjusted to 5.0 with the addition of sulfuric acid (11.4%) at 5.8 L/min. Once pH 5.0 was reached, the batch was digested for 20 minutes at 93° C. After digestion, the batch was filtered, washed to a conductivity of 1500 pS and was spray dried. The dry product was air milled to a median particle size of approximately 3 μm.

Pigment G

463 L of water and 183 L of sodium silicate (30%, 2.5 MR) were added to a 400-gallon reactor and heated to 85° C. with stirring at 75 RPM. Once 85° C. was reached, the pH was adjusted to 7.5 with the addition of sulfuric acid (11.4%) at 3.2 L/min. Once pH 7.5 was reached, sodium silicate (30%, 2.5 MR) and sulfuric acid (11.4%) were simultaneously added at 1.3 L/min and 1.6 L/min, respectively, for 30 minutes. After 30 minutes had passed, the flow of silicate was stopped and the pH was adjusted to 5.0 with continued addition of sulfuric acid (11.4%) at 1.6 L/min. Once pH 5.0 was reached, the batch was digested for 30 minutes at 93° C. After digestion, the batch was filtered, washed to a conductivity of 1500 μS and was spray dried. The dry product was air milled to a median particle size of approximately 1 μm.

Comparative Pigments H, I, and J

For comparison, commercial products denoted herein as Pigment H, Pigment I and Pigment J were purchased. Rona Sphere spherical silica (Pigment H) and Rona Sphere LDP spherical silica treated with titanium dioxide, and iron oxide (Pigment I) were obtained from Rona/EM Industries, Hawthorne, N.Y.; and Spheron N-2000 spherical silica (Pigment J) was obtained from Presperse, Inc., Somerset, N.J. Properties of both the inventive and comparative pigments are summarized in Table 1 below.

TABLE 1

| Pigment | | MPS | |
|---|---|---|---|
| No. | Pigment Description | (μm) | Oil absorption (ml/100 g) |
| A | Sodium aluminosilicate | 10.9 | 108 |
| B | Sodium aluminosilicate | 8.7 | 102 |
| C | Sodium aluminosilicate | 1.9 | 109 |
| D | Sodium magnesium | 0.9 | 133 |
| | aluminosilicate | | |
| E | Sodium aluminosilicate | 1.4 | 170 |
| F | Silica | 3.1 | 201 |
| G | Silica | 1.2 | 227 |
| H | Rona Sphere | 2.0 | 41 |
| I | Rona Sphere LDP | 5.6 | 25 |
| J | Spheron N-2000 | 4.6 | 14 |

Median particle size (MPS) was determined using a Model LA-910 laser light scattering instrument available from Horiba Instruments, Boothwyn, Pa. A laser beam is projected through a transparent cell which contains a stream of moving particles suspended in a liquid. Light rays which strike the particles are scattered through angles which are inversely proportional to their sizes. The photodetector array measures the quantity of light at several predetermined angles. Electrical signals proportional to the measured light flux values are then processed by a microcomputer system to form a multi-channel histogram of the particle size distribution.

Oil absorption, using linseed oil, was determined by the rubout method. This method is based on a principle of mixing oil with a pigment by rubbing with a spatula on a smooth surface until a stiff putty-like paste is formed. By measuring the quantity of oil required to have a paste mixture, which will curl when spread out, one can calculate the oil absorption value of the pigment—the value which represents the volume of oil required per unit weight of pigment to saturate the pigment sorptive capacity. Calculation of the oil absorption value was done as follows:

$$\begin{matrix}Oil absorption = \frac{ml oil absorbed}{weight of pigment , grams} \times 100 \\ = ml oil / 100 g pigment\end{matrix}$$

In the following examples, several of the wrinkle hiding pigments described above were compared in several cosmetic formulations by measuring the total and diffuse transmittance of a thin film of the formulation and also evaluating in vivo application of the formulations to human subjects with digital photography.

The integrating sphere evaluation method described below is similar to the one that was first described by Nakamura et al, with the exception that the optical properties of thin films of the pigment in actual cosmetics compositions were measured. Total and diffuse transmittance measurements were made with a Perkin Elmer Lambda 35 UV/Visible spectrophotometer equipped with a Labsphere RSA-PE-20 reflectance spectroscopy accessory. Test formulations contained a wrinkle hiding pigment while the control contained the same ingredients as the test formulation with the exception of the wrinkle hiding pigment. A small amount (˜0.25 g) of the wrinkle hiding formulation was applied to a glass slide. A thin film was made by dragging an RDS laboratory coating rod (RDS08) in a single pass over the slide. Once the film was dry, total and diffuse transmittance measurements were made by scanning the visible spectrum. Since the transmittance values varied only slightly across the visible spectrum, for simplicity, only the values at 550 nm were reported. An empty sample holder and the Spectralon® reflectance standard was used as a blank prior to making both total and diffuse transmittance measurements. It is important to note that small variations in the procedure, such as those affecting the quantity of each ingredient used to prepare the formulations or those that can change the thickness of the films, etc., will alter the transmittance results. Therefore, it is important to be as consistent as possible when making formulations, preparing films and to make comparisons of the relative performance of one pigment versus another under the same batch/testing conditions. In accordance with the work performed by Nakamura, the pigment compositions exhibiting the highest diffuse transmittance values were most effective at blurring wrinkles when applied to the skin.

Empirical evaluations were performed and digital pictures were taken of those evaluated subjects for archival purposes [pictures were taken with a Canon Digital Rebel (6.3 mega-pixel) with a 100 mm macro lens or an 18 to 55 mm lens]. The left hand or forehead of a subject was used as the evaluation site for such empirical evaluations. Typically, a focusing aid was applied to the testing area to provide sufficient contrast for the camera to focus, such as a black dot applied with a marker or a small multi-colored sticker. The focus dot/sticker also served as a reference point to achieve the proper camera-subject alignment in subsequent pictures. A photo was taken of the skin area before the application of any test formulations for comparative purposes. A small amount of the formulation was applied to the testing area, was rubbed in thoroughly and allowed to dry before empirical evaluations and such archival digital pictures were taken. If the focus dot/sticker appeared blurry in a photo, the image was discarded because the blurriness in the picture was a result of an improperly focused camera and not the wrinkle hiding pigment. The testing area was washed thoroughly with soap and water and dried before the evaluation of subsequent formulations.

### Example 1

Several wrinkle hiding pigments were incorporated into a water-based cosmetic formulation. The formulation consisted of two parts. Part I was comprised of deionized water (14.0%) and wrinkle hiding pigment (5.0%). Part II was comprised of deionized water (75.0%), GERMABEN II (1.0%, ISP/Sutton, Chatham, N.J.) and Structure XL (5.0%, National Starch, Bridgewater, N.J.). The ingredients in Part I of the formulation were mixed together. Part II of the formulation was prepared by mixing together approximately 90% of the water and Structure XL and mixing for 15 minutes. The remaining portion of the water and the Germaben II was then added and Part II was then slowly heated to 50° C. Once 50° C. was reached, Part I was slowly added to Part II with mixing. The batch was then slowly cooled to room temperature. The formulations were analyzed with the integrating sphere (Table 2) and digital camera evaluation methods.

TABLE 2

| | Total Transmittance | Diffuse Transmittance |
|---|---|---|
| Pigment | 550 nm (%) | 550 nm (%) |
| None | >85 | 10.0 |
| A | >85 | 7.0 |
| B | >85 | 42.2 |
| C | >85 | 52.5 |
| H | >85 | 40.0 |
| I | >85 | 12.0 |
| J | >85 | 19.6 |

While all samples exhibited total transmittance values greater than 85%, the diffuse transmittance values for Pigment C were found to be the highest out of the samples tested. It was also found that by reducing the particle size from 10.9 μm to 1.9 μm, the diffuse transmittance values could be increased from approximately 7% (Pigment A) to 52.5% (Pigment C). Next, the water-based formulation containing Pigment C was compared to the water-based formulation containing Pigment H by applying the formulations (separately) to the same location of a subject's hand. Photos were taken and then rated by a panel of 25 persons based upon the extent the wrinkles in each image were blurred. Greater than 95% of the panelists chose Pigment C as a more effective wrinkle blurring pigment than Pigment H. It was also found that the Pigments with the highest diffuse transmittance values exhibited the most effective wrinkle blurring performance.

### Example 2

Several wrinkle hiding pigments were incorporated into an oil-in-water emulsion cosmetic formulation. The formulation consisted of three parts. Part I was comprised of deionized water (64.9%), Veegum (1.0%, RT Vanderbilt, Norwalk, Conn.) and Germaben II (0.1%, ISP/Sutton, Chatham, N.J.). Part II was comprised of RitaPro 300 (5.5%, Rita, Crystal Lake, Ill.), Cetiol OE (4.5%, Cognis, Cincinnati, Ohio), Cetoil 868 (3.0%, Cognis, Cincinnati, Ohio) and Finsolv TN (1.0%, Finetex, Spencer, N.C.). Part III was comprised of deionized water (15.0%) and wrinkle hiding pigment (5.0%). Parts I and II were heated to 80° C., combined and homogenized at approximately 2000 rpm for 15 minutes. The batch was then mixed with a paddle style mixer and Part III was added. After sufficient mixing, the batch was slowly cooled to room temperature. The formulations were then analyzed with the integrating sphere (Table 3) and digital camera evaluation methods.

TABLE 3

| | Total Transmittance | Diffuse Transmittance |
|---|---|---|
| Pigment | 550 nm (%) | 550 nm (%) |
| None | >85 | 21.0 |
| B | >85 | 52.0 |
| C | >85 | 66.6 |
| D | >85 | 63.0 |
| E | >85 | 63.0 |
| F | >85 | 68.4 |
| G | >85 | 66.0 |
| H | >85 | 41.0 |
| I | >85 | 29.5 |
| J | >85 | 33.5 |

The total transmittance of all pigments tested was greater than 85%. It was found that the diffuse transmittance of Pigments C, D, E, F and G were greater than that of Comparative Pigments H, I and J. Reduction of the particle size from 10.9 μm to 1.9 μm resulted in an increase in the diffuse transmittance values from 52.0% (Pigment B) to 66.6% (Pigment C). Next, the visibility of wrinkles were evaluated before and after application of the oil-based formulation containing Pigment C by applying the formulation to subject's forehead. It was found that the pigments with the highest diffuse transmittance values exhibited the most effective wrinkle blurring performance. Empirically, the viewed results of the subject's forehead were consistent with the diffuse transmittance values as well.

### Example 3

Several wrinkle hiding pigments were incorporated into a water-in-silicone emulsion cosmetic formulation. The formulation consisted of three parts. Part I was comprised of RHEOPEARL® KL (2.2%, US Cosmetics, Dayville, Conn.), Dow Corning 193 Surfactant (1.9%, Dow Corning, Midland, Mich.), Trivent OC-G (5.0%, Trivent, Monmouth Junction, N.J.) and Dow Corning 5225C Formulation Aid (10.0%, Dow Chemical, Midland, Mich.). Part II was comprised of Dow Corning 345 Fluid (25.0%, Dow Corning, Midland, Mich.). Part III was comprised of deionized water (49.9%), sodium chloride (1.0%) and wrinkle hiding pigment (5.0%). The wrinkle hiding pigment was slowly added and completely mixed with the other ingredients in Part III. Part I was heated to 85° C. and stirred until all the solids were dissolved. Once dissolved, Part I was then slowly cooled to 50° C. and at which time Part II was added with stirring. Once homogeneous, Part III was slowly added and the batch was sufficiently mixed and slowly cooled to room temperature. The formulations were then analyzed with the integrating sphere (Table 4) and empirical (digital camera) evaluation methods.

TABLE 4

| | % Total Transmittance | % Diffuse Transmittance |
|---|---|---|
| Pigment | 550 nm | 550 nm |
| None | >90 | 10.5 |
| B | >90 | 50.3 |
| C | >90 | 70.6 |
| H | >90 | 38.1 |
| I | >90 | 48.8 |
| J | >90 | 27.5 |

The total transmittance was greater than 90% for all pigments tested. The diffuse transmittance value for Pigment C (70.6%) was the highest of all pigments tested. The reduction in particle size from 10.9 μm to 1.9 μm resulted in an increase in the diffuse transmittance values from 50.3% (Pigment B) to 70.6% (Pigment C). Next, the silicone-based formulation containing Pigment C was compared to the silicone-based formulation containing Pigment H by applying the formulations (separately) to the same location of a subject's hand. The digital pictures clearly showed that the formulation containing Pigment C blurred wrinkles more effectively than the formulation containing Pigment H. It was also noticed that the pigments with the highest diffuse transmittance values exhibited the most effective wrinkle blurring performance. Again, the empirical views confirmed such results as well.

While the invention will be described and disclosed in connection with certain preferred embodiments and practices, it is in no way intended to limit the invention to those specific embodiments, rather it is intended to cover equivalent structures structural equivalents and all alternative embodiments and modifications as may be defined by the scope of the appended claims and equivalence thereto.

We claim:

1. A method of providing optical blurring of a skin surface comprising the steps of
a) providing a cosmetic formulation for application to skin comprising an appropriate cosmetic vehicle and at least one type of pigment, wherein said pigment is a material that is comprised of a plurality of particles that are predominately non-spherical and three-dimensional in nature, wherein the median particle size of such plurality of particles is from 1 to 20 μm, and wherein said plurality of particles exhibits an average oil absorption capacity of from about 10 to 300 ml/100 g; and
b) applying such a cosmetic formulation to a selected area of skin.

2. The method of claim 1 wherein the median particle size of the plurality of particles within the cosmetic formulation is from about 1 to 10 μm.

3. The method of claim 2 wherein said median particle size is from about 1 to 5 μm.

4. The method of claim 3 wherein said median particle size is from about 1 to 3 μm.

5. The method of claim 1 wherein the average oil absorption capacity of the plurality of particles within the cosmetic formulation is from about 100 to 240 ml/100 g.

6. The method of claim 1 wherein said cosmetic formulation is present within a form selected from the group consisting of a solid, a gel, a paste, a lotion, a cream, a spray, a loose powder, a pressed powder, and a liquid.

7. The method of claim 2 wherein said cosmetic formulation is present within a form selected from the group consisting of a solid, a gel, a paste, a lotion, a cream, a spray, a loose powder, a pressed powder, and a liquid.

8. The method of claim 3 wherein said cosmetic formulation is present within a form selected from the group consisting of a solid, a gel, a paste, a lotion, a cream, a spray, a loose powder, a pressed powder, and a liquid.

9. The method of claim 4 wherein said cosmetic formulation is present within a form selected from the group consisting of a solid, a gel, a paste, a lotion, a cream, a spray, a loose powder, a pressed powder, and a liquid.

10. The method of claim 5 wherein said cosmetic formulation is present within a form selected from the group consisting of a solid, a gel, a paste, a lotion, a cream, a spray, a loose powder, a pressed powder, and a liquid.
